(19) United States
(12) Patent Application Publication
(10) Pub. No.: US 2003/0045577 A1
(43) Pub. Date: Mar. 6, 2003

(54) Method for preventing infectious respiratory diseases

(76) Inventor: Maher N. Madhat, Lexington, KY (US)

Correspondence Address: MAHER MADHAT, 3305 GRASMERE DRIVE, LEXINGTON, KY, 40503, US

(21) Appl. No.: 09/930,399

(22) Filed: Aug. 15, 2001

(51) Int. Cl. A61K 31/20; A61K 31/13

(52) U.S. Cl. 514/558; 514/560; 514/662

(57) ABSTRACT

A method and composition for treating patients with viral infection with pharmaceutical agents is disclosed. In one embodiment, the virus is influenza A and the pharmaceutical agent is rimantadine or amantadine. The methods of the present invention can be used as a way of eradicating influenza virus topically by direct administration of a dose—nasally—that is much lower than an oral therapeutic dose. Thereby avoiding the side effects associated with oral administration. Consequently, the low dose of the drug administered by this method will be well tolerated among age groups

### FIELD OF INVENTION

The present invention pertains to the prevention of viral disease in humans utilizing nasal pharmaceutical compositions. In particular, the present invention pertains to the method of the prevention of influenza A viral infection in humans with rimantadine or amantadine or their water soluble or insoluble salts thereof administered nasally.

### BACKGROUND OF THE INVENTION

Epidemic influenza continues to be associated with significant morbidity in general population and mortality in the elderly and other risk patients. Although the case fatality rate averages less than 0.01%, tens of thousands of deaths occur each year.

Amantadine is a drug developed in 1960s with diverse uses ranging from prevention of influenza A to the treatment of patients with Parkinson's disease [Aoki and Sitar, Clin Pharm 14:35-51, 1988]. Amantadine hydrochloride is a well known compound commercially available as Symmetral. Available dosage forms of Symmetrel® are soft gelatin capsules and oral syrup [U.S. Pat. No. 3,310,469 assigned to Du Pont describes composition-containing amantadine]. Elimination of amantadine is primarily through renal clearance by both glomerular filtration and tubular secretion. Amantadine accumulates in patients with renal dysfunction. Therefore, doses must be reduced in such patients to avoid toxicity [Aoki and Sitar, 1988]. Further, amantadine as an antiviral agent-of nucleic acid derivative type-is likely to bring about side effects such as deterioration of liver function, mutagenicity, sub-acute toxicity, teratogenicity and a decrease in reproductive efficiency [Virology—published by Raven press, pp 323-348, 1985].

This invention relates to a method and pharmaceutical compositions containing rimantadine or amantadine or salts thereof, suitable for nasal topical application as a prophylaxis against influenza A virus. These drugs are currently administered orally at relatively high dose (˜200 mg daily), which are associated with adverse reactions such as nausea, dizziness, and insomnia.

Influenza virus infection is acquired by a mechanism involving the transfer of virus-containing respiratory secretions from an infected individual through various passages such as sneezing, coughing, talking, or breathing to a healthy individual [Robert F. Betts, in Principles and Practice of Infectious Disease, fourth edition, Churchill Livingstone]. Once the virus is deposited on the epithelium respiratory tract it can attach to and penetrate columnar epithelial cells and start infecting adjacent and nearby cells leading to an onset of virus shedding or an onset of illness within 19-72 hours. Therefore, nasal application of the drug defuses the entry of the virus to the lung. Consequently the present invention represents a novel prophylactic measure to control influenza A infection. While an understanding of the precise mechanism is not necessary to carry out the methods of the present invention, it is believed these drugs block events in late viral uncoating or early transcription [Skehel et al. 1978, J.Gen.Virol 38:97-110].

### SUMMARY OF THE INVENTION

The present invention acts as a prophylaxis against influenza A virus infection utilizing pharmaceutical agents. In one embodiment, the pharmaceutical agent of the present invention is rimantadine or amantadine and the viral infection is influenza A. One embodiment of the present invention contemplates

a. providing

i. individual exposed to influenza virus

ii. a therapeutically effective dose of said rimantadine or amantadine to said individual under such conditions such that symptoms of said infection is reduced.

The present invention is limited by the method of administration

Furthermore, the present invention is limited to acute influenza infection

The present invention provides an antiviral agent in a topical, non-oral form

The present invention uses a much lower dose to reach an effective therapy.

### DETAILED DESCRIPTION OF THE INVENTION

The high occurrence of side effects has prompted the investigator to search for another way to fight the virus at the port of entry into the human body, which should be efficacious in the treatment of influenza virus. Rimantadine and amantadine are currently administered orally at a relatively high dose that is associated with systemic toxicity/side effects and not well tolerated. Viral replication event occurs mainly in the nasal cavity, which then proceeds to the lung. Therefore the present invention is a prophylaxis meant to fight the virus by topical delivery of rimantadine or amantadine to the nasal cavity at doses much lower than oral doses.

The active ingredient will ordinarily be present in an amount of about 0.05-95% by weight based on total weight of the composition. The active ingredient can be administered to a mammal at a dose between 0.01-0.7 mg/kg while the preferred dose is 0.07-0.3 mg/kg.

Composition of the active ingredients can be administered topically to the nasal cavity by preparing a suitable formulation of the active ingredient by procedures well known to those skilled in the art. Preferably the formulations are prepared with nontoxic pharmaceutically acceptable ingredients. These ingredients are known to those skilled in the preparation of nasal dosage forms and some of these can be found in REMINGTON'S PHARMACETUCAL SCIENCES. 17TH edition, 1985 a standard reference in the field. The choice of suitable carriers is highly dependent upon the exact nature of the nasal dosage form desired, e.g. solutions, suspensions, ointments, gels or nasal metered dose inhalers. Nasal dosage forms generally contain large amounts of water in addition to the active ingredient, except for metered dose inhalers with propellant. Minor amounts of other ingredients such as pH adjusters, emulsifiers or dispersing agents, preservatives, surfactants, jelling agents, or buffering agents may also be present. Preferably the nasal dosage form should be isotonic with nasal secretion. If desired, a sustained nasal compositions e.g. sustained release gels, can be readily prepared. The sustained release form formed preferably by employing the desired drug in one of its relatively insoluble forms, such as a long chain carboxylic acid salt. The carboxylic acid portion of the salt preferably contains 10 to 20 carbon atoms.

### EXAMPLE 1

The following example provides a method for preparing rimantadine or amantadine water-insoluble fatty acid salts:

The fatty acid salts of the present invention can be prepared by treating aqueous solutions of rimantadine hydrochloride or amantadine hydrochloride with aqueous solutions of the commercially available water-soluble salts, such as sodium salts, of the fatty acids.

As an example, amantadine stearate salt is prepared as follows:

Amantadine stearate:

Sodium stearate (0.01 mole) was dissolved in hot water. An aqueous solution containing amantadine hydrochloride (0.011 mole) was added drop wise to the sodium stearate solution by stirring while heated. The suspension formed was cooled to room temperature and the precipitate was filtered and dried (m.p. 115-118 C). Elemental analysis calculated for C₂₈H₅₃NO₂: C, 77.18%; H, 12.26%; N, 3.21%. Found C, 77.26%; H, 12.28%; N, 3.18%.

### EXAMPLE 2

This example provides a procedure for preparing a sustained release gel of the water-insoluble fatty acid salts of rimantadine or amantadine:

Eighty (80 g) of water was heated to 80° C., and 3.0 g of methycellulose were added, with stirring. The resulting mixture was allowed to stand at room temperature for 3 hours to form a gel. Twenty-two (22 g) of amantadine stearate was suspended in 20 g of water. The resulting suspension was added to the gel and mixed thoroughly. The resultant was a viscous preparation or gel, which was adjusted to isotonicity with sodium chloride. The obtained sustained release composition thus contained 22 mg of amantadine stearate in 0.1 mL.

### EXAMPLE 3

As an example of a nasal solution composition of this invention includes:

| | | |
|---|---|---|
| Rimantadine or | 22 g | active drug |
| amantadine salt | | |
| Citric acid | | as needed to create a buffered solution |
| Sodium citrate | | as needed to create a buffered solution |
| Methylcellulose | | as a suspending agent if a suspension is |
| | | used |
| Methylparaben | 100 mg | as a preservative |
| Propylparaben | 20 mg | as a preservative |
| Sodium chloride | | as needed for tonicity |
| Hydrochloric acid | | as needed to adjust pH |
| or Sodium hydroxide | | |
| Purified water | | to a total preparation volume of 100 mL |

The formulation in this invention may be varied to include: (1) other acids and bases to adjust the pH; (2) other tonicity imparting agents such as sorbitol, glycerin, and dextrose; (3) other antimicrobial preservatives such as parahydroxybenzoic acid esters (such as sorbate, benzoate, propionate), chlorobutanol, phenylethyl alcohol, and benzalkonium chloride; (4) other viscosity imparting agents such as sodium carboxymethylcellulose, microcrystalline cellulose, polyvinylpyrrolidone, polyvinyl alcohol and other gums; (5) stabilizing agents such as antioxidants, like bisulfite and ascorbate, and metal chelating agents such as sodium editate.

The above formulation can be administered as drops, sprays, gels, ointments, aerosols, or by other intranasal dosage forms. Optionally, the delivery system can be a unit dose delivery system. The volume of solution or suspension delivered per dose can be anywhere from 20 to 400 microliters, and preferably between 50 and 150 microliters. Delivery systems of these various dosage forms can be dropper bottles, plastic squeeze units, atomizers, nebulizers or pharmaceutical aerosols in either unit doses or multidose packages.

I claim:

1. A method of preventing and treating influenza infection in humans consisting essentially of:
a. providing:
i. an individual who may have a potential of acquiring the disease by exposure
ii. said exposure comes from a patient having symptoms of influenza, and
iii formulations consisting of rimantadine or amantadine either in a soluble or insoluble drug form; and
b. administering a therapeutically effective dose of said formulation to said individual under conditions such that the probability of acquiring said influenza infection is reduced; and

2. The method of claim 1, wherein said formulation of rimantadine or amantadine salt is in a form selected from the group consisting of a solution, gel, metered dose inhaler, and ointment.

3. The method according to claim 1 wherein rimantadine or amantadine is administered as a suspension of a water insoluble long chain carboxylic acid salt, the carboxylic acid portion of the salt contains from 10 to 20 carbon atoms.

4. The method according to claim 3 wherein the long-chain carboxylic acid salts is stearate, palmitate or myristate.

5. The method of claim 3, wherein said formulation is in a form selected from the group consisting of a nasal gel, ointment, and metered dose inhaler
